(19) United States
(12) Patent Application Publication
(10) Pub. No.: US 2006/0246129 A1
(43) Pub. Date: Nov. 2, 2006

(54) Composition for use in treatment of sleep problems and method for same

(76) Inventors: Nikos Michalis Linardakis, Salt Lake City, UT (US); Norman Reg Wainwright, Falmouth, MA (US)

Correspondence Address: AMIN LAW, LLC, 217 N. JEFFERSON ST., SUITE 100, CHICAGO, IL, 60661, US

(21) Appl. No.: 11/413,648

(22) Filed: Apr. 28, 2006

Related U.S. Application Data
(60) Provisional application No. 60/676,682, filed on Apr. 29, 2005.

(51) Int. Cl. A61K 36/84; A61K 31/714; A61K 31/7048; A61K 31/455; A61K 31/405; A61K 31/198; A61K 31/525; A61K 31/51; A61K 31/4415

(52) U.S. Cl. 424/451; 424/682; 424/733; 514/27; 514/52; 514/251; 514/276; 514/350; 514/356; 514/561; 514/456; 514/419

(57) ABSTRACT

The present invention provides a non-addictive composition for the treatment of sleep disorders and problems including melatonin, valerian root extract, an antioxidant component, and a vitamin complex component. The present invention further provides a non-addictive composition for the treatment of sleep disorders and problems including melatonin, valerian Root Extract, proanthocyanidin, vitamin E, vitamin B₃, vitamin B₆, vitamin B₁₂, L-theanine, and magnesium. The present invention also provides a method for treating sleep disorders or problems of a human including administering a non-addictive composition for the treatment of sleep disorders or problems comprising a melatonin, valerian root extract, an antioxidant component, and a vitamin complex component; and causing the circadian rhythm of the human to be normalized and regulated, causing the sleep patterns of the human to be healthy and normal, and causing reduction of stress and anxiety in the human.

### CROSS REFERENCE TO RELATED APPLICATIONS

This application claims the benefit of U.S. provisional application No. 60/676,682, filed on Apr. 29, 2005.

### FIELD OF THE INVENTION

The present invention relates to a composition for use in a nutritional product, dietary supplement, pharmaceutical compound, or food composition, or for use as a food additive wherein such composition is used to treat sleep problems safely, effectively, and naturally. The composition includes a hormone component, an herbal extract component, an antioxidant component, a vitamin complex component, an amino acid component, and a mineral component. The present invention also relates to a method of treatment for sleep disorders or problems.

### BACKGROUND OF THE INVENTION

Nearly half of all adults including people under stress, students, shift workers, travelers, and people with a predisposition to worry experience sleep disorders or problems due to stress, anxiety, little amount of sleep and inconsistent sleep patterns associated with a fast-paced and over-worked lifestyle. Sleep disorders and problems exist when a regular biological clock known as a circadian rhythm or sleep-wake cycle, which is based on a 24-hour, day/night cycle, cannot be attained or maintained. The circadian rhythm is controlled and maintained by the hypothalamus and specifically the suprachiasmatic nucleus located in the brain. The circadian rhythm assists with several physiological and neurological processes in the body including but not limited to the sleep-wake cycle, daytime behavior, hormone (for example, growth hormone and cortisol stress hormone) release, and dietary consumption and satiety. The circadian rhythm also affects behavior and lack of adequate sleep can cause irritability, inability to concentrate, drowsiness, and decreased physical endurance. Over time, if the circadian rhythm is not regulated, these issues can continue leading to constant inattention, difficulty concentrating, passive and aggressive behavior, poor motor control, and a tendency to engage in addictive behavior such as smoking.

Sleep disorders and problems include insomnia, restless leg syndrome, problems inducing and maintaining sleep, sleep deprivation, jet lag, and other alertness and sleep related problems. Insomnia includes difficulty falling asleep, difficulty staying asleep, and waking up too early. Insomnia can be mild (only occasional bouts of sleeplessness lasting a couple nights) to acute (consistent sleeplessness lasting weeks to months to years). According to the National Sleep Foundation, nearly 6 out of 10 people experience at least one symptom of insomnia a few nights a week. Restless leg syndrome is a sleep disorder that causes tingling, numbness, tiredness, cramping, and aching in the legs. Many factors can lead to insomnia, sleep deprivation, inconsistent sleep patterns and other sleep problems including long work hours, persistent stress, diet, use of stimulants or alcohol, lack of exercise, psychiatric problems, excessive noise or light, anxiety, inability to relax, and the general hectic lifestyle of society. Sleep deprivation, insomnia, and inconsistent sleep patterns not only leave a person tired, but cause problems such as health, attitude, and performance problems, and the inability to function properly.

The most common treatment for sleep disorders or problems is the use of pharmacological therapies; the most popular being hypnotic agents such as benzodiazepines, antidepressants, barbiturates, and other drugs or alcohol. The current therapies for sleep disorders or problems have addictive potential.

Currently the therapies used to treat sleep disorders and problems have common short-term side effects such as amnesia, mood changes, hangover effects, and decreased cognitive function.

Currently treatments for sleep disorders and problems have undesirable and adverse interactions with other drugs and alcohol.

In view of the above, there is a need or a desire for a treatment for sleep disorders or problems which is natural, not addictive or does not cause physical dependency.

There is also a need or desire for a treatment for sleep disorders or problems which does not affect moods, create a hangover effect or drowsiness, or decrease cognitive ability.

There is further a need or desire for a treatment for sleep disorders or problems which do not adversely or undesirably interact with other drugs or alcohol.

### SUMMARY OF THE INVENTION

In response to the challenges discussed above, a composition to treat sleep disorders or problems safely, effectively, and naturally and is designed to normalize or regulate the circadian rhythm, and promote healthy and normal sleep patterns, falling into and staying asleep, and awakening refreshed has been developed.

The present invention is a non-addictive composition for the treatment of sleep disorders or problems including a hormone component, an herbal extract component, an antioxidant component, a vitamin complex component, an amino acid component, and a mineral component. The sleep disorders or problems include insomnia, restless leg syndrome, problems inducing and maintaining sleep, sleep deprivation, jet lag, and other alertness and sleep related problems. The present invention is also used for the purpose of quitting smoking. The form of the composition of the present invention may be a capsule, tablet, powder, gel, or liquid.

In one embodiment of the present invention the non-addictive composition for the treatment of sleep disorders or problems may have melatonin, valerian root extract, proanthocyanidin, Vitamin E, Vitamin B₃, Vitamin B₆, Vitamin B₁₂, L-theanine, and Magnesium.

The present invention also relates to a method for treating sleep disorders or problems. The method includes administering a non-addictive composition for the treatment of sleep disorders or problems comprising a hormone component, a herbal extract component, an antioxidant component, a vitamin complex component, an amino acid component, and a mineral component; and causing the circadian rhythm of the human to be normalized and regulated, causing the sleep patterns of the human to be healthy and normal, and causing reduction of stress and anxiety in the human. The method may further cause the prevention of female symptoms from pre-menopausal, peri-menopausal, or post-menopausal hormonal changes affecting sleep cycles. The method may also further assist in weight management and the maintenance of a healthy satiety cycle. The method may also further assist with quitting smoking. Suitably, the non-addictive composition may be administered in a single daily dose taken prior to attempting sleep.

These and other embodiments are more fully described in connection with the drawings and detailed description.

### DETAILED DESCRIPTION OF THE INVENTION

The present invention relates to a composition for use in a nutritional product, dietary supplement, pharmaceutical compound, or food composition, or for use as a food additive wherein such composition is used to treat sleep problems safely, effectively, and naturally. The present invention also relates to a method of treatment for sleep disorders or problems.

One embodiment of the present invention relates to a composition including a hormone component, an herbal extract component, an antioxidant component, a vitamin complex component, an amino acid component, and a mineral component.

The composition components have therapeutic properties which together treat common sleeping problems or disorders including insomnia, restless leg syndrome, problems inducing and maintaining sleep, sleep deprivation, jet lag, and other alertness and sleep related problems. The composition is designed to normalize and regulate the circadian rhythm, to promote healthy and normal sleep patterns by reducing stress and anxiety, and to help falling into and staying asleep, thereby helping to awaken refreshed. The composition also assists with quitting smoking. The composition further promotes the prevention of female symptoms from pre-menopausal, peri-menopausal, or post-menopausal hormonal changes affecting sleep cycles. The composition also assists in weight management, and the maintenance of a healthy satiety cycle. The composition is not habit forming, does not produce a hangover effect, maintains cognitive ability, and does not create adverse or undesirable interactions with other drugs. The composition components may be included in any of their commonly available forms.

The hormone component acts by suppressing the neuron activity which affects sleep induction and promotes relaxation, deep and calm sleep, a regulated circadian rhythm, and a reduction of stress. Suitably, the hormone component may be melatonin. Melatonin is a hormone normally and naturally secreted by the pineal gland; however, in individuals experiencing sleep disorders or problems the natural production is found in decreased concentrations. Melatonin acts by suppressing the suprachiasmatic nucleus (SCN) neuron activity which affects sleep induction and promotes relaxation, deep and calm sleep, a regulated circadian rhythm, and a reduction of stress.

The herbal extract component promotes staying asleep by relaxing and calming the nervous system. Suitably, the herbal extract component may be valerian root extract. The valerian root extract promotes staying asleep through its relaxation, and calming of the nervous system effects.

The antioxidant component is effective in supporting the immune system and its natural resistance to bacterial infections and reducing oxidative stress that occurs during sleep. Suitably, the antioxidant component may be a proanthocyanidin. An antioxidant is a scavenger which searches for dangerous cellular invaders, protects the body from oxidation and other stresses, and promotes the immune system with any deficits and its natural resistance to bacterial infections. Proanthocyanidins are antioxidants found in colorful fruits, including, but not limited to, wild blueberries and grapes. Proanthocyanidins are water-soluble, highly bioabsorbable phytochemicals which are effective in improving circulation, improving brain function, supporting the immune system, inhibiting cellular damage, and slowing the destructive power of free radicals to help reduce oxidative stress during sleep.

The vitamin complex component increases the efficiency of the hormone component, induces sleep, and acts as a cofactor in important biochemical reactions of the body. Suitably, the vitamin complex component may be vitamin E and a vitamin B complex. The vitamin E increases the efficiency of the hormone component in the induction of sleep. The vitamin B complex induces sleep and acts as a cofactor in important biochemical reactions of the body. Further suitably, the vitamin B complex may be vitamin B₃, B₆, and B₁₂.

The amino acid component promotes relaxation and reduces stress. Suitably, the amino acid component may be L-theanine. L-theanine can be found naturally in plants including, but not limited to, the green tea plant. L-theanine alters the levels of neurotransmitters of the brain and generates alpha-waves in the brain which promotes mental relaxation and the induction of sleep, and reduces stress.

The mineral component affects neuromuscular relaxation and calming, and is essential for achieving and maintaining normal sleep patterns. Suitably, the mineral component may be magnesium. A magnesium deficiency, which occurs due to certain conditions, medications, and diets of processed, nutrient-depleted foods, can cause nervousness which prevents the induction of sleep. Magnesium affects neuromuscular relaxation and calming, and is essential for achieving and maintaining normal sleep patterns.

In another embodiment, the composition of the present invention includes a hormone component, an herbal extract component, an antioxidant component, a vitamin complex component, an amino acid component, and a mineral component wherein the hormone component is melatonin; the herbal extract component is valerian root extract; the antioxidant component is a proanthocyanidin; the vitamin complex component is vitamin E, vitamin B₃, Vitamin B₆, and Vitamin B₁₂; the amino acid component is L-theanine; and the mineral component is magnesium.

In a further embodiment, a daily dose administration of the composition of the present invention includes:

melatonin approximately less than 1.0% by weight;

valerian root extract from approximately 20.0 to 60.0% by weight;

proanthocyanidins from approximately 1.0-9.0% by weight;

vitamin E from approximately 1.5-6.0% by weight;

vitamin B₃ from approximately 1.5-5.0% by weight;

vitamin B₆ from approximately 1.5-5.0% by weight;

vitamin B₁₂ approximately less than 2.0% by weight;

L-Theanine from approximately 3.0-25.0% by weight; and

magnesium from approximately 5.0-20.0% by weight.

The present invention also relates to a method of treating sleep disorders or problems involving the administration to a human of the composition of the present invention which causes the circadian rhythm of the human to be normalized and regulated, the sleep patterns of the human to be healthy and normal, and reduces the stress and anxiety in the human. In one preferred embodiment, the administration of the composition of the present invention is a single daily dose taken prior to attempting to sleep. The administered composition of the present invention may be in any suitable oral form including, but not limited to, a capsule, tablet, powder, gel, or liquid. In another preferred embodiment, the administered composition of the present invention is a food product, drug, functional food, food additive, beverage, dietary supplement or pharmaceutical compound.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

We claim:

1. A non-addictive composition for quitting smoking and the treatment of sleep disorders or problems, comprising:
melatonin;
valerian root;
an antioxidant component; and
a vitamin complex component.

2. The composition of claim 1 wherein the sleep disorders or problems are insomnia, restless leg syndrome, problems inducing and maintaining sleep, sleep deprivation, jet lag, and other alertness and sleep related problems.

3. The composition of claim 1 further comprising an amino acid component and a mineral component.

4. The composition of claim 1 wherein the antioxidant component is proanthocyanidin.

5. The composition of claim 1 wherein the vitamin complex component is vitamin E and a vitamin B complex.

6. The composition of claim 5 wherein the vitamin B complex is vitamin B₃, B₆, and B₁₂.

7. The composition of claim 3 wherein the mineral component is magnesium.

8. The composition of claim 3 wherein the amino acid component is L-theanine.

9. The composition of claim 1 wherein the form of the composition is a capsule, tablet, powder, gel, and liquid.

10. A non-addictive composition for quitting smoking and the treatment of sleep disorders or problems, comprising:
melatonin;
valerian root extract;
proanthocyanidin;
vitamin E;
vitamin B₃;
vitamin B₆;
vitamin B₁₂;
L-theanine; and
magnesium.

11. A non-addictive composition for quitting smoking and the treatment of sleep disorders or problems, in a daily dose treatment, comprising:
melatonin approximately less than 1.0% by weight;
valerian root extract from approximately 20.0 to 60.0% by weight;
proanthocyanidins from approximately 1.0-9.0% by weight;
vitamin E from approximately 1.5-6.0% by weight;
vitamin B₃ from approximately 1.5-5.0% by weight;
vitamin B₆ from approximately 1.5-5.0% by weight;
vitamin B₁₂ approximately less than 2.0% by weight;
L-Theanine from approximately 3.0-25.0% by weight; and
magnesium from approximately 5.0-20.0% by weight.

12. A method for quitting smoking and treating sleep disorders or problems of a human comprising the steps of:
administering a non-addictive composition comprising melatonin, valerian root,
an antioxidant component, and a vitamin complex component; and
causing the circadian rhythm of the human to be normalized and regulated,
causing the sleep patterns of the human to be healthy and normal and causing reduction of stress and anxiety in the human.

13. The method of claim 12 wherein the sleep disorders or problems are insomnia, restless leg syndrome, problems inducing and maintaining sleep, sleep deprivation, jet lag, and other alertness and sleep related problems.

14. The method of claim 12 wherein the composition administered further comprises an amino acid component and a mineral component.

15. The method of claim 12 wherein the antioxidant component is proanthocyanidin.

16. The method of claim 12 wherein the vitamin complex component is vitamin E and a vitamin B complex.

17. The method of claim 16 wherein the vitamin B complex is vitamin B₃, B₆, and B₁₂.

18. The method of claim 14 wherein the mineral component is magnesium.

19. The method of claim 14 wherein the amino acid component is L-theanine.

20. The method of claim 12 further comprising promoting the prevention of female symptoms from pre-menopausal, peri-menopausal, or post-menopausal hormonal changes affecting sleep cycles.

21. The method of claim 12 further comprising assisting the human in weight management and the maintenance of a healthy satiety cycle.

22. The method of claim 12 wherein the administering of the non-addictive composition is in a single daily dose taken prior to attempting sleep.
